(19) United States
(12) Patent Application Publication
(10) Pub. No.: US 2015/0159132 A1
(43) Pub. Date: Jun. 11, 2015

(54) TECHNOLOGY FOR CELL TRANSDIFFERENTIATION

(71) Applicant: Fluidigm Corporation, South San Francisco, CA (US)

(72) Inventors: NIANZHEN LI, South San Francisco, CA (US); Marc Unger, South San Francisco, CA (US)

(21) Appl. No.: 14/568,597

(22) Filed: Dec. 12, 2014

Related U.S. Application Data
(63) Continuation of application No. PCT/US2013/045848, filed on Jun. 14, 2013.
(60) Provisional application No. 61/659,927, filed on Jun. 14, 2012.

(51) Int. Cl. C12N 5/079; C12M 3/06; B01L 3/00; G01N 33/50; C12Q 1/68

(52) U.S. Cl. CPC C12N 5/0618; G01N 33/5026; C12Q 1/6881; B01L 3/5027; C12M 23/16; C12N 2506/02; C12N 2501/65; C12Q 2600/158; B01L 2300/0829; B01L 2200/0647

(57) ABSTRACT

This invention provides technology for transdifferentiating cells from one cell type to another. The cells are cultured with one or more vector-free gene regulator oligonucleotides concurrently or in succession, and then harvested when cell markers or the morphology of the culture shows that transdifferentiation is complete. Suitable gene regular oligonucleotides include microRNAs and messenger RNAs that encode a differentiation factor. Conditions for transdifferentiation can be optimized by dividing cells into different culture chambers of a microfluidic device. Cells are cultured with different additives in each chamber, and then compared. Transdifferentiated cells produced according to this invention can provide a consistent source of tissue for use in regenerative medicine.

### PRIORITY

This application is a U.S. continuation of International Patent Application No. PCT/US2013/045848, which was filed on Jun. 14, 2013 and published as WO 2013/188748 on Dec. 19, 2014, and which claims the priority benefit of U.S. Provisional Patent application 61/659,927, filed Jun. 14, 2012. The aforelisted previous applications are hereby incorporated herein by reference in their entirety for all purposes.

### FIELD OF THE INVENTION

The invention relates generally to the field of microfluidic devices, cell culture, and cell modification. More specifically, it provides devices, reagents, and methods for changing the phenotype and gene expression of a cell from one cell type to another.

### BACKGROUND

The ability to transform cells from one lineage into another lineage is a burgeoning area in research science and regenerative medicine. Another area being developed is the technology of microfluidic devices.

Lesher-Perez S C et al, Biotechnol J. 2013 Feb.; 8(2):180-91 discuss microfluidic systems for manipulating pluripotent stem cells. Vyawahare S et al., Chem Biol. 2010 Oct. 29; 17(10):1052-65, discuss miniaturization and parallelization of biological and chemical assays in microfluidic devices. Sisakhtnezhad S et al., Cell Tissue Res. 2012 Jun.; 348(3):379-96, describe transdifferentiation as a cell and molecular reprogramming process. Published patent application US 2006/0160066 A1 (S N Bhatia et al., University of California) refer to cellular microarrays for screening differentiation factors.

There is a need for improved devices and methods for testing and causing transdifferentiation of certain cell types and for otherwise screening products using cultured cells.

### SUMMARY OF THE INVENTION

One aspect of this invention is a method for transdifferentiating a cell from a first cell type to a second cell type. The cell is cultured in medium containing an oligonucleotide composition comprising one or more vector-free gene regulator oligonucleotides so that the cell is transdifferentiated from the first cell type to the second cell type.

The oligonucleotide composition may comprise one or more vector-free microRNA, such as miR-9, miR-9*, and miR-1. Alternatively or in addition, the composition may comprise a messenger RNA encoding a differentiation factor, or an agent that causes a differentiation factor to be expressed by the cell itself. Other components may be included, such as lipids for promoting transfection of the first cell type by oligonucleotides in the composition. In one example, the cell is contacted with the composition during at least three periods over at least four days with media changes in between.

The method of transdifferentiation can be put into practice in a multi-step transdifferentiation protocol. This typically comprises administering a first composition to the cell, said composition comprising a first vector-free gene regulator oligonucleotide; culturing the cell in the presence of the first composition; withdrawing the first composition from the cell; administering a second composition to the cell, the composition comprising a second vector-free gene regulator oligonucleotide that was not present in the first composition; and then culturing the cell in the presence of the second composition. The culture cycle may be repeated iteratively two, three, four, or more than four times.

Each composition used in one or more of the cycles may (but does not necessarily) include an agent that promotes differentiation of a first cell type to the second cell type or to an intermediate cell type. Such agents may include one or more of the following in any combination: oligonucleotides such as microRNA or mRNA, biological factors (such as proteins), recombinantly produced equivalents thereof, and chemical compounds known to have the desired effect. A composition used in one or more of the cycles may (but does not necessarily) include an agent that stops or regulates differentiation.

Transdifferentiation can be evaluated by assessing morphology or gene expression of the cell or the progeny thereof after transdifferentiation: for example, using a PCR assay for mRNA in the cell. Expression of one or more markers selected from NCAM1, DCX, MAP-2, TUBB3, SCN1A, PTBP-2, and PTBP-1 can be used to monitor transdifferentiation of a fibroblast to a neural cell. The transdifferentiation can be performed in a microfluidic device. The process can be concluded by assessing the cells while in the device, or the cells can be harvested from the microfluidic device for assessment and/or further culturing or processing. In one example, the method may be performed on a cell population comprising a plurality of cells of the first cell type such that substantially a majority of the cells of the first cell type are transdifferentiated into cells of the second cell type.

Another aspect of the invention is a method for evaluating conditions for transdifferentiating a cell from a first cell type to a second cell type in a microfluidic device. The method comprises loading a plurality of cells into separate reaction sites of a microfluidic device; simultaneously culturing the cells in the separate reaction sites under culture conditions that vary among the reaction sites in one or more parameters over a predetermined range; and then determining whether cells in at least one of the reaction sites have transdifferentiated from the first type to the second type.

Using this method, transdifferentiation protocols can be developed and optimized by selecting the conditions that are substantially the same as the conditions used in a particular reaction site in the microfluidic device. Then reaction conditions are adjusted to improve or continue the transdifferentiation over a predetermined range, and the experiment is repeated. Parameters that can be varied and optimized include selection of one or more oligonucleotides from a library of gene regulator. Other parameters that can be varied and optimized include the time of culture with a differentiation factor, the temperature, humidity, partial pressure of a gas, and the combination, concentration or ratio of the differentiation factors used.

For example, the user may vary the selection or concentration of a first gene regulator oligonucleotide cultured with to the cells in a first stage of transdifferentiation, and/or the selection or concentration of a second gene regulator oligonucleotide cultured with the cells in a second or subsequent stage. Such gene regulator oligonucleotides may be one or more microRNAs selected from miR-9, miR-9*, miR-124, mi-R1 miR-21, miR-22, mi-R23, miR-122, miR-122a, miR-148, microRNAs in the let-7b family, microRNAs of other differentiation factors, in any combination.

The reaction sites may be arranged in the microfluidic device in a grid pattern such that one of the parameters may be varied in each row of the grid, and a second of the parameters may be varied in each column of the grid. The microfluidic device may have a multiplexer by which different reagents may be administered to different cells at different times. When in use, each reaction site may contain a single or a plurality of cells. The cells at each reaction site may be sequentially cultured with a plurality of different factors. The microfluidic device may have a multiplexer by which different reagents may be administered to different cells at different times.

Another aspect of the invention is a microfluidic device configured to identify or verify effective reagents that transform cells of a first type into cells of a second type in a multi-step transdifferentiation protocol. The device has a plurality of units, each unit comprising a chamber configured for cell culture; a delivery means for introducing one or more cells into the culture chamber; a delivery means for introducing a first composition to cells in the culture chamber; a waste means for withdrawing the first differentiation composition from the culture chamber; a delivery means for introducing a second composition to cells into the culture chamber; a waste means for withdrawing the second differentiation composition from the culture chamber; and a means for analyzing and/or recovering cells in the culture chamber after culturing.

Each composition may comprise one or more differentiation factors that are different from each other, present at a different composition, or accompanied by different adjunct factors or compounds. Each delivery means may be (for example) a channel optionally having a valve fluidly connected to a reservoir containing the element to be introduced into the chamber. Each waste means may be (for example) a channel optionally with a valve fluidly connected to a waste container.

The units in the device can be interconnected so that different first compositions and/or different second compositions are introduced into each of the culture chambers. The units can be arranged in a grid pattern such that a culture additive or parameter may be varied in each row of the grid, and a second culture additive or parameter may be varied in each column of the grid. The device can be configured for optically assessing the morphology of cells in each of the culture chambers, and/or a sensing means for a biological marker expressed by or present on the second cell type.

During use, a plurality of the culture chambers contains cells, and the factor delivery means contain various compositions for separate delivery to each of the culture chambers. The first and second compositions for each unit may contain one or more differentiator factors selected from a vector-free gene regulator oligonucleotide, a microRNA, a messenger RNA encoding a differentiation factor, or an oligonucleotide that affects expression of a differentiation factor by the cell.

Another aspect of the invention is the use of any suitably configured microfluidic device for transdifferentiating cells from one type to another according to a method of this invention. Another aspect of the invention is a cell population or tissue produced according to a transdifferentiate method of this invention, which has been rendered suitable for administration to a patient in need thereof for the purpose of therapy. Another aspect of the invention is the use of such cells or tissues in regenerative medicine.

Other aspects of the invention will be apparent from the description that follows.

### BRIEF DESCRIPTION OF THE DRAWINGS

FIG. 1(A) depicts a cell-culture microfluidic device mounted on a chip carrier. The carrier provides input and output reservoirs and channels, pneumatic control that operates valves of the delivery system, and precise environmental control. FIG. 1(B) is a flow chart showing how different combinations of various factors can be precisely delivered to culture chambers in the microfluidic device at programmed time points. FIG. 1(C) provides an illustration in which differentiation agents and conditions are varied amongst different culture chambers on the device.

FIG. 2 provides details of the design of such a microfluidic device. In this example, 32 cell culture chambers can be individually treated with any combination of eight input factors.

FIG. 3 shows the morphology of cells obtained at different times following the first round of transfection with the agents indicated on the vertical axis.

FIG. 4 shows the morphology of cells at the end of the protocol treated with one or two of the microRNAs alone, rather than all three.

FIG. 5 compares cells treated with different microRNA combinations by phase contrast, and by immunohistochemical staining of the same field for beta-III tubulin.

FIG. 6(A) depicts a single-cell capture site in a microfluidic device. FIG. 6(B) and FIG. 6(C) show analysis of a gene expression pattern of a plurality of cell samples. (The vertical annotations below each pixel in FIG. 6(B) are laboratory designations for particular markers, and are provided for purposes of illustration. Knowledge of each particular designation is not required for understanding or practice of the invention.)

FIG. 7 shows combinatorial effects of microRNAs in multi-factorial experiments done on a microfluidic device to improve culture compositions used in transdifferentiation.

### DETAILED DESCRIPTION OF THE INVENTION

This invention provides technology for transdifferentiating cells from one cell type to another. The cells are cultured with one or more vector-free gene regulator oligonucleotides concurrently or in succession, and then harvested when cell markers or the morphology of the culture shows that transdifferentiation is complete. Suitable gene regular oligonucleotides include microRNAs and messenger RNAs that encode a differentiation factor. Conditions for transdifferentiation can be optimized by dividing cells into different culture chambers of a microfluidic device. Cells are cultured with different additives in each chamber, and then compared. Transdifferentiated cells produced according to this invention can provide a consistent source of tissue for use in regenerative medicine.

### Transdifferentiation Factors and Conditions

Transdifferentiation, also known as lineage reprogramming or direct conversion, is a process where cells convert from one differentiated cell type to another without undergoing an intermediate pluripotent state or progenitor cell type. Transdifferentiation has been proposed as an approach for disease modeling, drug discovery, gene therapy and regenerative medicine. Related publications include the following:
Vierbuchen T, Ostermeier A, Pang Z P, Kokubu Y, Südhof T C, Wernig M. Direct conversion of fibroblasts to functional neurons by defined factors. Nature. 2010 Feb. 25; 463(7284):1035-41.
Pang Z P, Yang N, Vierbuchen T, Ostermeier A, Fuentes D R, Yang T Q, Citri A, Sebastiano V, Marro S, Südhof T C, Wernig M. Induction of human neuronal cells by defined transcription factors. Nature. 2011 May 26; 476(7359):220-3.
Yoo A S, Sun AX, Li L, Shcheglovitov A, Portmann T, Li Y, Lee-Messer C, Dolmetsch R E, Tsien R W, Crabtree G R. MicroRNA-mediated conversion of human fibroblasts to neurons. Nature. 2011 Jul. 13; 476(7359):228-31.
Ambasudhan R, Talantova M, Coleman R, Yuan X, Zhu S, Lipton S A, Ding S. Direct reprogramming of adult human fibroblasts to functional neurons under defined conditions. Cell Stem Cell. 2011 Aug. 5; 9(2):113-8.
Caiazzo M, Dell'Anno M T, Dvoretskova E, Lazarevic D, Taverna S, Leo D, Sotnikova T D, Menegon A, Roncaglia P, Colciago G, Russo G, Carninci P, Pezzoli G, Gainetdinov R R, Gustincich S, Dityatev A, Broccoli V. Direct generation of functional dopaminergic neurons from mouse and human fibroblasts. Nature. 2011 Jul. 3; 476(7359):224-7.

Pfisterer U, Kirkeby A, Torper O, Wood J, Nelander J, Dufour A, Björklund A, Lindvall O, Jakobsson J, Parmar M. Direct conversion of human fibroblasts to dopaminergic neurons. Proc Natl Acad Sci USA. 2011 Jun. 21; 108(25):10343-8
Son E Y, Ichida J K, Wainger B J, Toma J S, Rafuse V F, Woolf C J, Eggan K. Conversion of mouse and human fibroblasts into functional spinal motor neurons. Cell Stem Cell. 2011 Sep. 2; 9(3):205-18.
Marro S, Pang Z P, Yang N, Tsai M C, Qu K, Chang H Y, Südhof T C, Wernig M. Direct lineage conversion of terminally differentiated hepatocytes to functional neurons. Cell Stem Cell. 2011 Oct. 4; 9(4):374-82.
Qiang L, Fujita R, Yamashita T, Angulo S, Rhinn H, Rhee D, Doege C, Chau L, Aubry L, Vanti W B, Moreno H, Abeliovich A. Directed conversion of Alzheimer's disease patient skin fibroblasts into functional neurons. Cell. 2011 Aug. 5; 146(3):359-71.
Kim K S. Converting human skin cells to neurons: a new tool to study and treat brain disorders? Cell Stem Cell. 2011 Sep. 2; 9(3):179-81.
Warren L, Manos P D, Ahfeldt T, Loh Y H, Li H, Lau F, Ebina W, Mandal P K, Smith Z D, Meissner A, Daley G Q, Brack A S, Collins J J, Cowan C, Schlaeger T M, Rossi D J. Highly efficient reprogramming to pluripotency and directed differentiation of human cells with synthetic modified mRNA. Cell Stem Cell. 2010. 7(5): 618-30.
   Inclusion of a reference in the list above or anywhere in this disclosure should not be construed as an indication that the reference constitutes prior art to the claimed invention.

A fundamental challenge for developing cell transdifferentiation protocols is finding the right culture conditions for cell expansion, differentiation and reprogramming

One aspect of this invention is technology for screening and identifying differentiation factor and factor combinations for transdifferentiating or de-differentiating cells from one cell type to another. The technology comprises a microfluidic device adapted for culturing cells in the presence of such factors while varying one or more culture parameters across a pre-determined range. The technology also provides methodology for assessing the effect of such factors and parameters on the cell morphology, phenotype or function in situ.

Microfluidic technologies, coupled with biocompatible materials, employ precise control of cell microenvironments, facilitate studies of multi-factorial combinations, and enable the development of robust, reproducible and chemically defined cell culture systems.

By using the microfluidic screening technology of this invention, it has been discovered that cell transdifferentiation can be effected with unexpected precision, control, and efficiency using vector-free gene regulator oligonucleotides. These include but are not limited to microRNA, as described in the sections that follow.

### Screening Transdifferentiation Factors in a Microfluidic Device

This invention provides a microfluidic chip and an automated instrument that can culture cells in chambers on a chip for an extended period of time and deliver multiple combinations of different factors to cells. Differentiation reagents can be automatically multiplexed in desired combinations and ratios at various predefined time points. Cells can also be harvested from the chip for continued off-chip culturing and single-cell genomic analysis.

Microfluidic technology for culturing cells has been described elsewhere. See Gómez-Sjöberg R, Quake S R, et al., Versatile, fully automated, microfluidic cell culture system. Anal Chem. 2007 Nov. 15; 79(22):8557-63. Zhong J F et al., A microfluidic processor for gene expression profiling of single human embryonic stem cells. Lab Chip. 2008 Jan.; 8(1):68-74. Glotzbach J P et al., An information theoretic, microfluidic-based single cell analysis permits identification of subpopulations among putatively homogeneous stem cells. PLoS One. 2011; 6(6):e21211. Sanchez-Freire V et al., Microfluidic single-cell real-time PCR for comparative analysis of gene expression patterns. Nat Protoc. 2012 Apr. 5; 7(5):829-38. U.S. Pat. No. 7,378,280 and US 2011/0166044 A1: Apparatus and methods for conducting assays and high throughput screening. US 2010/0255471 A1: Single cell gene expression for diagnosis, prognosis and identification of drug targets. The aforelisted publications are hereby incorporated herein by reference in their entirety for all purposes.

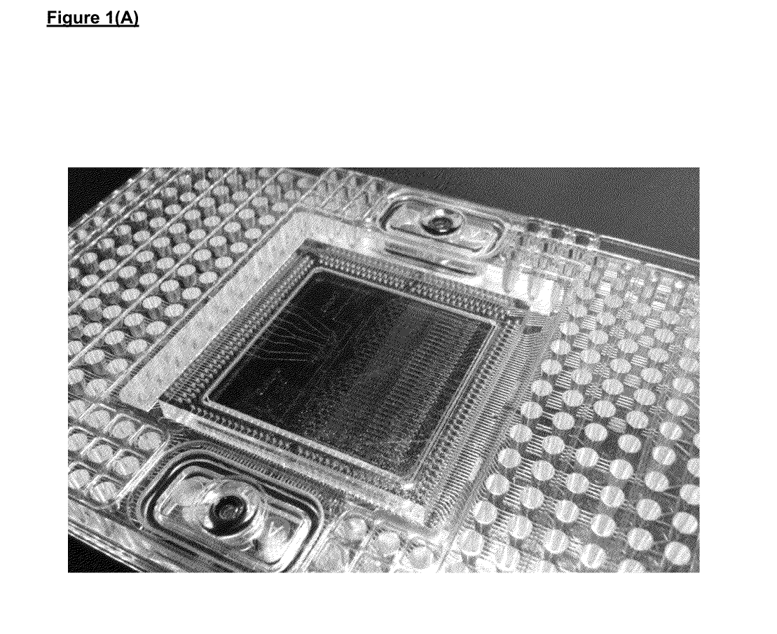

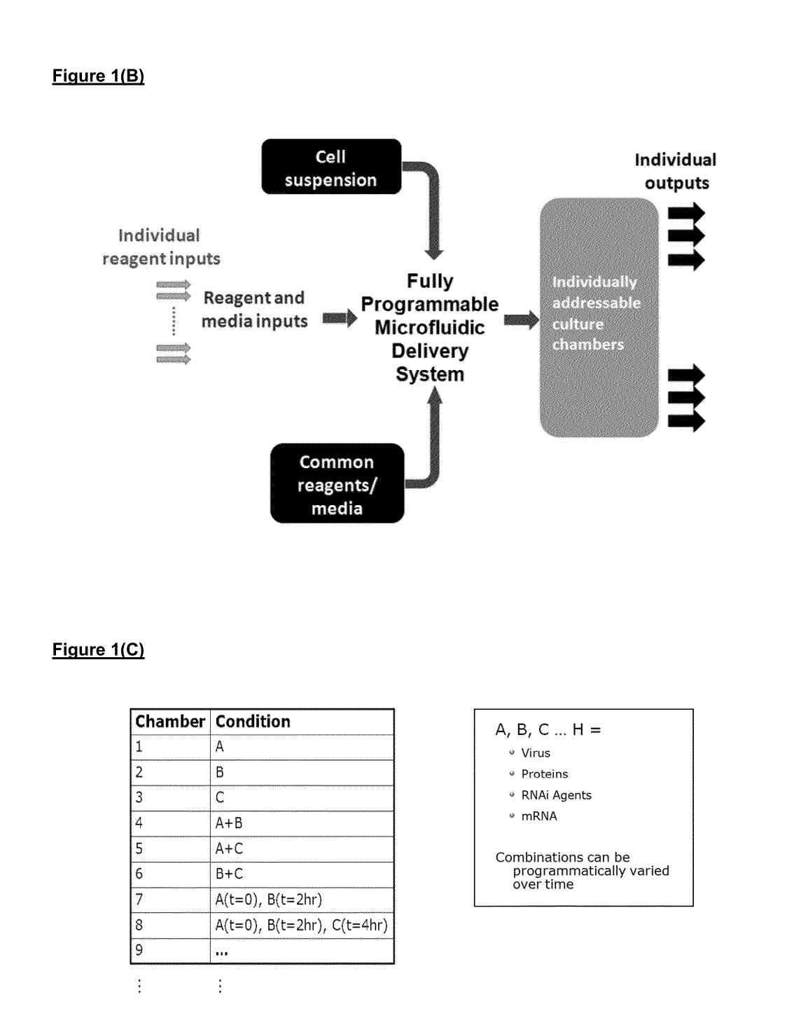

FIG. 1 depicts a prototype automated controller instrument and a cell-culture chip using multilayer Soft Lithography manufacturing processes. The chip is mounted on a plastic carrier which serves as the Input/Output mechanism: FIG. 1(A). The controller seals to the chip carrier, provides pneumatic control for the microfluidic delivery system and precise environmental control including temperature, humidity, and gas mixture: FIG. 1(B). Multiple different combinations of various factors can be precisely delivered to cells on the chip at programmed time points: FIG. 1(C) provides examples of how differentiation agents and conditions can be varied amongst different culture chambers on the chip.

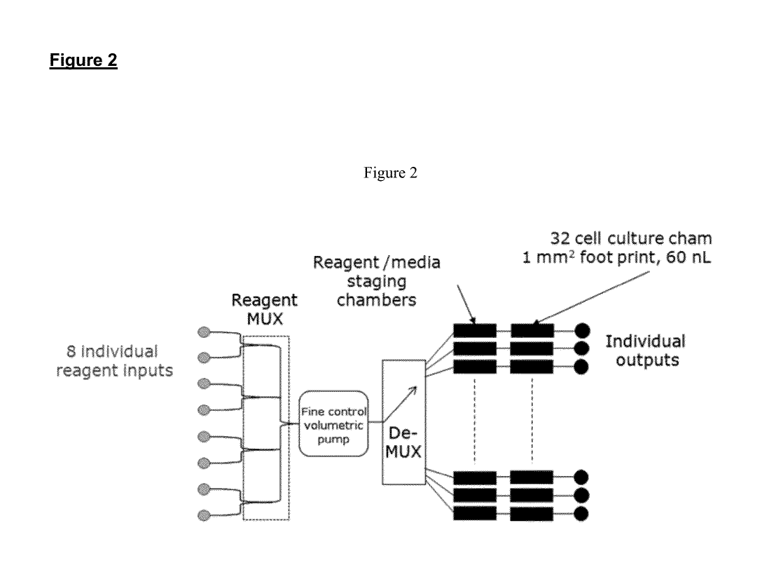

FIG. 2 provides details of the design of such a microfluidic device. The cell-culture chip features 32 cell culture chambers which can be individually treated with any combination of 8 input factors—simultaneously or on a preprogrammed schedule. These chambers (one mm² footprint, 60 nL volume) can be individually addressed through the main multiplexer or MUX. A peristaltic pump allows gentle delivery of precise amounts of reagents to the cells. At the end of an experiment or at predefined time points, the cells can be exported from individual chambers into individual output wells using appropriate resuspension reagents.

### Features of the Device and Screening Process

In general terms, the devices for use in accordance with the screening methodology of this invention are microdevices having reaction chambers for culturing one or more cells under conditions that allow cells to conduct their usual metabolic activity, and adapt according to transdifferentiation factors included in the medium. The reaction chamber will be configured for cell populations of the desired size: either single cells, or populations of at least 10, 50, 250, 1000, 5000, 25,000, 100,000 or more than 100,000 cells each. The reaction chamber will be provided with an appropriate gaseous mixture (typically comprising O₂ and CO₂) in accordance with the cell's needs, and a source of nutrient medium. It may be fluidly connected to a cell source for loading, an output for sampling, a source of fresh medium, and a source of regulatory oligonucleotides and/or other potential differentiation factors to be tested.

The device will have a plurality of such reaction chambers and assemblies so that a matrix of different factors and/or reaction conditions may be screened simultaneously. The device may comprise at least 10, 50, 250, 1000, 5000, 25,000, 50,000 or more than 100,000 such reaction chambers or assemblies, typically arranged in a regular pattern such as a grid. Flow into and out of the chambers will typically be controlled through a series of valves such as gated valves with flexible membranes through another layer of the device, for each chamber individually and/or for a plurality of chambers in different columns or rows. Where a plurality of different parameters are varied across all the reaction sites on the device, each reaction site may be prepared individually using a multiplexer that can be used to provide the adjusted variable of each parameter from each of the sourced components.

Screening is conducted by culturing cells in different reaction sites under conditions that vary among the reaction sites in one, two, three, four, or more than four parameters. One parameter is culturing with oligonucleotides such as microRNAs, and optionally other factors, to promote transdifferentiation. Such parameters may include specificity and/or concentration and/or incubation time with one, two, three, or more than three oligonucleotides and zero, one, two, three, or more than three other potential differentiation factors in any combination, and/or other variables such as culture temperature, gas partial pressure, or humidity

Assessment of the effect may comprise an evaluation of the morphology of the cells in the reaction sites. The microfluidic device is adapted to be substantially transparent around and about the reaction site. The transdifferentiated cells are viewed by the operator or by an imaging device in communication with a processing device. Morphology of the cells at each reaction site can then be compared with morphology of the starting cell population, the originating cell type, and/or the target cell type. The proportion of cells having particular morphological features can be quantified. The effect of the transdifferentiation protocol in each reaction site can then be compared across the device to determine which conditions are optimal or more effective in achieving the desired result.

Assessment of the effect may also comprise an evaluation of cell markers. This can be done, for example, by immunohistochemistry using specific antibody, for example, conjugated with a marker such as a fluorescent dye. The cells in each well are sampled, or the staining is done on the entire population in the well in situ. After the staining reaction is complete, expression of the marker can be assessed by determining the overall intensity of fluorescent staining in the well, electronically counting cells present in each well that are stained as a proportion of the total number of cells present, or flowing the cells out of the reaction site through a cell detector or sorter in a microfluidic channel that can assess each cell individually.

Assessment of the effect may also comprise an evaluation of gene expression at the mRNA level. This can be done, for example, by polymerase chain reaction (PCR) that generates a detectably labeled product. The assay can be done on the cell population as a whole, or the population can be sampled for assaying separately, optionally in another chamber in the device (in situ). This disclosure provides technology whereby gene expression may be evaluated in a single cell sampled from a transdifferentiation reaction.

Improved Transdifferentiation Protocols using Vector-Free Gene Regulator Oligonucleotides

The screening technology of this invention contributed to the discovery that transdifferentiation of human or mammalian cells from one cell type to another can be effected under particular optimized conditions as described in this disclosure. This includes optimized use of gene regulator oligonucleotides that up or down regulate expression of differentiation factors.

This aspect of the invention provides a considerable improvement over previous approaches to transdifferentiation. Previous studies have described introducing transcription factors and microRNAs into cells for the purpose of transdifferentiation using a viral vector such as lentivirus. The use of viral vectors poses potential risk of DNA recombination and genome integration, hence are unsafe and unsuitable for clinical applications. Nevertheless, until the making of this invention, the use of viral vectors was seen as the primary choice because of the perceived inefficiency of other technology.

The data shown below demonstrate that human BJ fibroblasts can be transdifferentiated to cells having neuronal cell morphology and phenotypic markers by direct transfection with combinations of synthetic microRNA mimics. The transdifferentiation was highly efficient with high cell viability. The identities of cells were confirmed with immunostaining and gene expression profiling.

This discovery could not have been predicted in advance for several reasons. For example, a vector-free approach using microRNA would be expected to have low-efficiency. For example, iPS clones generated using microRNA are produced with a frequency of 2 colonies per 1×10⁵ human cells, which equals to 0.002% (Miyoshi et al., infra). This is considerably lower than what is obtained using viral vectors, which can show 700 to 5,000 fold higher efficiency See Warren et al., supra; and Anokye-Danso F et al., Highly efficient microRNA-mediated reprogramming of mouse and human somatic cells to pluripotency. Cell Stem Cell. 2011 Apr. 8; 8(4):376-88.

As a second example, using viral vectors provides a constitutive ongoing source of the differentiation oligonucleotide to the cell being transdifferentiated. Without viral transduction, repeated transfection would presumably be required to transdifferentiate cells from one cell type to another. The skilled reader would expect that this would be stressful and toxic to cells, especially for cells transitioning to or committing to a non-replicative cell type, such as a neural cell. Compared with fibroblasts, neurons are terminally differentiated and non-dividing, and they are much more delicate and sensitive to environmental factors; transfection into neurons have always been difficult; repeated transfection are even more stressful. In previous viral transduction methods, the cells were infected with virus only once, while they were still fibroblasts. With a single transduction, there is abundant cell death once cells start to show lineage conversion. Thus, the repeated transfection, especially transfection for cells already partly transdifferentiated, would be expected to be toxic to substantially all of the cells in the population.

Thus, using vector-free microRNA or other differentiation oligonucleotides for transdifferentiation is more challenging than using vector-free oligonucleotides for reprogramming—once the original cell type is transdifferentiated into cells of a mature line such as neural cells, they stop dividing. This creates a cell viability issue that is unique to the objective of switching to a non-proliferative cell type, rather than a dedifferentiated multipotent cell with proliferative capacity.

### Suitable Regulator Oligonucleotides for Transdifferentiation

Vector-free gene regulator oligonucleotides suitable for use for transdifferentiation in accordance with this invention include nucleic acids and nucleic acid analogs that specifically increase or decreases expression of one or more genes at the level of transcription or translation once transfected into a cell. This includes but is not limited to mRNA encoding one or more differentiation factors, and also includes RNAi, siRNA, oligonucleotide decoys and microRNA that inhibit transcription or translation of one or more differentiation factors.

Factors suitable for screening as agents for transdifferentiation in accordance with this invention are known and will be apparent to the reader. Additional factors may be identified in the future, using, for example, methods disclosed herein. Exemplary factors, for illustration and not limitation, are described in the following publications:
Zhou Q, Brown J, Kanarek A, Rajagopal J, Melton D A: In vivo reprogramming of adult pancreatic exocrine cells to beta cells. Nature 2008, 455:627-32.
Ambasudhan R, Talantova M, Coleman R, YuanX, Zhu S, Lipton S A, Ding S: Direct reprogramming of adult human fibroblasts to functional neurons under defined conditions. Cell Stem Cell 2011, 9: 113-8.
Yoo A S, Sun A X, Li L, Shcheglovitov A, Portmann T, Li Y, Lee-Messer C, Dolmetsch R E, Tsien R W, Crabtree G R: MicroRNA mediated conversion of human fibroblasts to neurons. Nature 2011, 476:228-31.
Qiang L, Fujita R, Yamashita T, Angulo S, Rhinn H, Rhee D, Doege C, Chau L, Aubry L, Vanti W B, Moreno H, Abeliovich A: Directed conversion of Alzheimer's disease patient skin fibroblasts into functional neurons. Cell 2011, 146:359-71.
Pfisterer U, Kirkeby A, Torper O, Wood J, Nelander J, Dufour A, Bjorklund A, Lindvall O, Jakobsson J, Parmar M: Direct conversion of human fibroblasts to dopaminergic neurons. Proc Natl Acad Sci USA 2011, 108:10343-8.

Pfisterer U, Wood J, Nihlberg K, Hallgren O, Bjermer L, Westergren-Thorsson G, Lindvall O, Parmar M: Efficient induction of functional neurons from adult human fibroblasts. Cell Cycle 2011, 10:3311-6.
11. Caiazzo M, Dell'Anno M T, Dvoretskova E, Lazarevic D, Taverna S, Leo D, Sotnikova T D, Menegon A, Roncaglia P, Colciago G, Russo G, Carninci P, Pezzoli G, Gainetdinov R R, Gustincich S, Dityatev A, Broccoli V: Direct generation of functional dopaminergic neurons from mouse and human fibroblasts. Nature 2011, 476:224-7.
Subramanyam D, Lamouille S, Judson R L, Liu J Y, Bucay N, Derynck R, Blelloch R: Multiple targets of miR-302 and miR-372 promote reprogramming of human fibroblasts to induced pluripotent stem cells. Nat Biotechnol 2011, 29:443-8.
Judson R L, Babiarz J E, Venere M, Blelloch R: Embryonic stem cell-specific microRNAs promote induced pluripotency. Nat Biotechnol 2009, 27:459-61.
Anokye-Danso F, Trivedi C M, Juhr D, Gupta M, Cui Z, Tian Y, Zhang Y, Yang W, Gruber P J, Epstein J A, Morrisey E E: Highly efficient microRNA-mediated reprogramming of mouse and human somatic cells to pluripotency. Cell Stem Cell 2011, 8:376-88.
Miyoshi N, Ishii H, Nagano H, Haraguchi N, Dewi D L, Kano Y, Nishikawa S, Tanemura M, Mimori K, Tanaka F, Saito T, Nishimura J, Takemasa I, Mizushima T, Ikeda M, Yamamoto H, Sekimoto M, Doki Y, Mori M: Reprogramming of mouse and human cells to pluripotency using mature microRNAs. Cell Stem Cell 2011, 8:633-8.
Subramanyam D, Lamouille S, Judson R L, Liu J Y, Bucay N, Derynck R, Blelloch R: Multiple targets of miR-302 and miR-372 promote reprogramming of human fibroblasts to induced pluripotent stem cells. Nat Biotechnol 29:443-8.
Sekiya S, Suzuki A: Direct conversion of mouse fibroblasts to hepatocyte-like cells by defined factors. Nature 2011, 475:390-3. Ieda M, Fu J D, Delgado-Olguin P, Vedantham V, Hayashi Y, Bruneau B G, Srivastava D: Direct reprogramming of fibroblasts into functional cardiomyocytes by defined factors. Cell 2010, 142:375-86.
   The aforelisted publications are hereby incorporated herein by reference in their entirety for all purposes.

In contrast to certain methods, in instances where the gene regulator oligonucleotide is used in a vector free composition, there will be no constitutive self-renewing source of the oligonucleotide inside the cell. This invention provides various elements by which the oligonucleotide may persist or rendered more efficient so as to compensate. Such elements include use of lipid-containing transfection agents to promote penetration of the oligonucleotide into the target cell; and adapted nucleotide acid backbone chemistry to resist breakdown and promote longer term effects. Oligonucleotides with altered backbone chemistry are reviewed, for example, by Geary R S. Expert Opin Drug Metab Toxicol. 2009 Apr.; 5(4):381-91. The user may also conduct repeated transduction with the oligonucleotide(s) over multiple time periods, optionally interspersed by periods in which the cells are washed with medium and/or cultured with fresh medium in the absence of oligonucleotide.

### Use of MicroRNAs

MicroRNAs (also referred to as miRNA or miR) are small endogenous RNA molecules (about 21 to 25 nucleotides) that regulate gene expression by targeting one or more mRNAs for translational repression or cleavage. Several thousand microRNAs have been identified in organisms as diverse as viruses, worms, and primates through cloning or computational prediction.

The structure of microRNA in vivo is described in Cai X et al. (2004) RNA 10 (12): 1957-66; and Kim Y K et al. (2007) EMBO J. 26 (3): 775-83. Assembly of microRNA from precursor oligonucleotides in vivo is described in Lin S L et al. (2005) Gene 356: 32-8; and Williams A E (2008) Cell. Mol. Life Sci. 65 (4): 545-62. Potential use of microRNA in medicine is discussed by Fasanaro, P. et al. (2010) Pharmacology & therapeutics 125 (1): 92-104; and Dimond, P F (2010) Genetic Engineering & Biotechnology News 30 (6): p. 1. A list of known microRNAs can be sourced from Ambros V et al. (2003) RNA 9 (3): 277-9; and Griffiths-Jones S et al. (2003) Nucleic Acids Res. 34: D 140-4. The publications listed in this paragraph are hereby each incorporated herein by reference in their entirety for all purposes.

Unless otherwise indicated, the term “microRNA” used in this disclosure with respect to agents for inducing or promoting transdifferentiation include microRNA mimics. The microRNAs may be human microRNAs and/or mimics thereof.

MicroRNA mimics with improved oligonucleotide chemistry suitable for use in the invention are commercially available and include Ambion® Pre-miR™ microRNA Precursor Molecules (Life Technologies, Grand Island, N.Y.). These are small, chemically modified double-stranded RNA molecules designed to mimic endogenous mature microRNAs. They are chemically similar to, but not identical to siRNAs. Other microRNA mimics include •mirVana™ microRNA mimics (Life Technologies), which are more specific than their predecessors due to inactivation of the star strand.

MicroRNAs that may be used for inducing transdifferentiation include miR-9 and miR-124 in the brain, miR-1 in muscle, and miR-122 in liver. To their respective cell types. Other microRNAs, such as the let-7 family, are broadly expressed across all differentiated tissues and, hence, are likely general stabilizers of the differentiated adult cell fate. See Melton C and Blelloch R (2010), MicroRNA Regulation of Embryonic Stem Cell Self-Renewal and Differentiation, Adv Exp Med Biol 2010, 95:105-17; and Shenoy A and Blelloch R (2012), MicroRNA induced transdifferentiation, F1000 Biology Reports 2012, 4:3.

miR-148 and let-7b are important in lens regeneration of adult newt; transfection of miR-148 and let-7b can induce dorsal pigment epithelial cells (PECs) cells transdifferentiate into lens cells. Nakamura K et al. (2010). miRNAs in Newt Lens Regeneration: Specific Control of Proliferation and Evidence for miRNA. Networking. PLoS ONE 5(8): e12058. miR-21, miR-22 and miR-122a are highly expressed after pancreatic acinar cells AR42J-B13 transdifferentiated into hepatocyte-like cells, and may be part of a mixture of factors induce transdifferentiation into pancreatic cells. Chen H-L et al., (2012). MicroRNA-22 Can Reduce Parathymosin Expression in Transdifferentiated Hepatocytes. PloS ONE 7(4): e34116.

The microRNA shown in Table 1 were used to illustrate the technology of this invention.

TABLE 1
Model microRNA for Transdifferentiation

hsa-miR-9-5p
Mature microRNA Sequence:
UCUUUGGUUAUCUAGCUGUAUGA (SEQ ID NO: 1)
Species: Human, Mouse, Rat
Mapped to miRBase Version: v18
Alias: hsa-miR-9 (v17)
hsa-miR-9*
Mature microRNA Sequence:
AUAAAGCUAGAUAACCGAAAGU (SEQ ID NO: 2)
Species: Human, Mouse, Rat
Mapped to miRBase Version: v18
Alias: hsa-miR-9* (v17)
hsa-miR-124-3p
Mature microRNA Sequence:
UAAGGCACGCGGUGAAUGCC (SEQ ID NO: 3)
Species: Human, Mouse, Rat
Mapped to miRBase Version: v18
Alias: hsa-miR-124 (v17)

Human neonatal BJ fibroblast cells were first seeded in multi-well cell-culture plates or Fluidigm microfluidic cell-culture chips in a fibroblast media (EMEM™ plus 10% FBS or DMEM/F12 medium plus 10% FBS), then switched to a transfection media (Opti-MEM™ medium supplemented with 4% FBS and 200 ng/mL B18R; Cell Biosciences). Cells were maintained in a 37 degree incubator with 5% CO₂ (5% or 20% O₂) (four well plates) or a Fluidigm® automated controller instrument that has environmental control for microfluidic chips. A variety of cell culture surfaces can be used, including non-coated cell culture plate, fibronectin, polyornithine-laminin or polylysine.

After one day, cells were transfected with microRNA mimics (and/or synthetic mRNAs encoding transcription factors) using lipid containing transfection reagents such as RNAiMAX™, Invitrogen™ Life Technologies, Grand Island, N.Y., or StemFect™, Stemgent, Cambridge Mass. Exemplary was StemFect™ RNA transfection kit (Stemgent Cat #00-0069), used according to manufacturer's directions.

MicroRNA mimics sold under the mark mirVana™ (specifically, hsa-miR-9, 9*, and 124, or combinations thereof were delivered to cells to convert fibroblasts to neurons. Controls were FAM-labeled negative control microRNA, or media alone (NTC: non-transfection control). For transfection regimens, for well-plate experiments cells were exposed to transfection complex for 4-6 hours every one to three days (optimal two days for enhanced cell viability and transfection efficiency) with media change in between. For on-chip experiments cells were exposed to transfection complex for 10-12 hours every two days with media change in between. The concentrations of combined microRNAs range from 12.5 nM to 25 nM.

Four days post-initial transfection media were switched to neuronal media (ScienCell, Carlsbad Calif.) supplemented with B18R, valproic acid and basic FGF. After 14 days, transfection was stopped, and cells were maintained in neuronal media plus 0.5 mM dbcAMP.

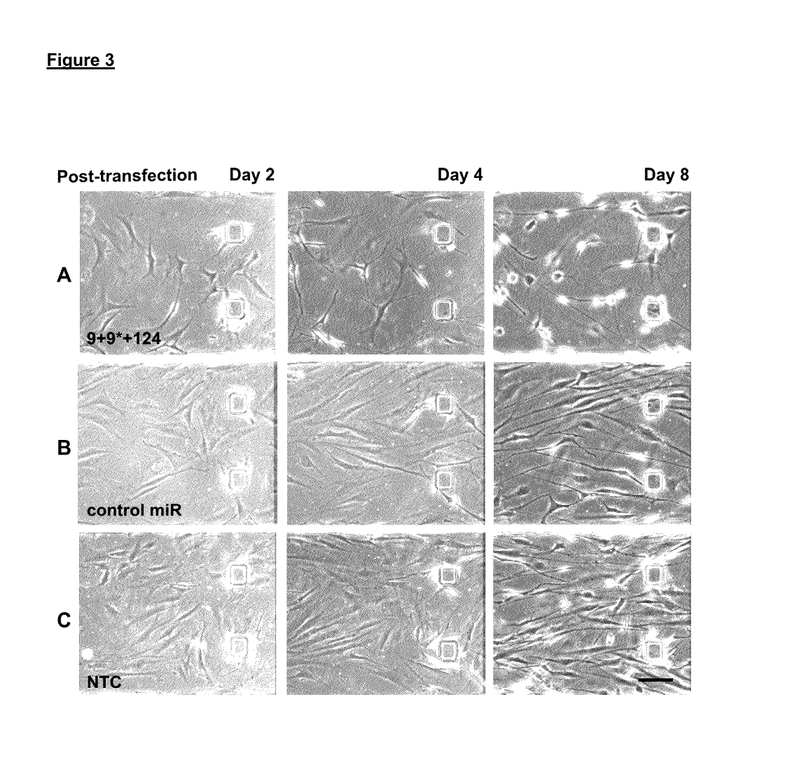

FIG. 3 is a series of phase-contrast micrographs showing the morphology of cells obtained at Day 2, Day 4, or Day 8 following the first round of transfection with the combination of microRNA 124/9/9*, with control microRNA, or with media alone. MicroRNAs 9, 9*, 124 or their combinations were delivered to different chambers to convert human BJ fibroblasts into neurons on chip. Row (B) shows the effect of FAM-labeled negative control microRNA. Row (C) shows the effect of media only (NTC: non-transfection control) Inhibition of cell proliferation by microRNA was observed as early as day 2. A few cells started to show neuronal-like morphology as early as day 4; after day 7, many cells (˜50-80%) assumed neuronal morphology. Scale bar: 100 mm. Note the two bright squares in each image are structures of posts in the cell culture chambers.

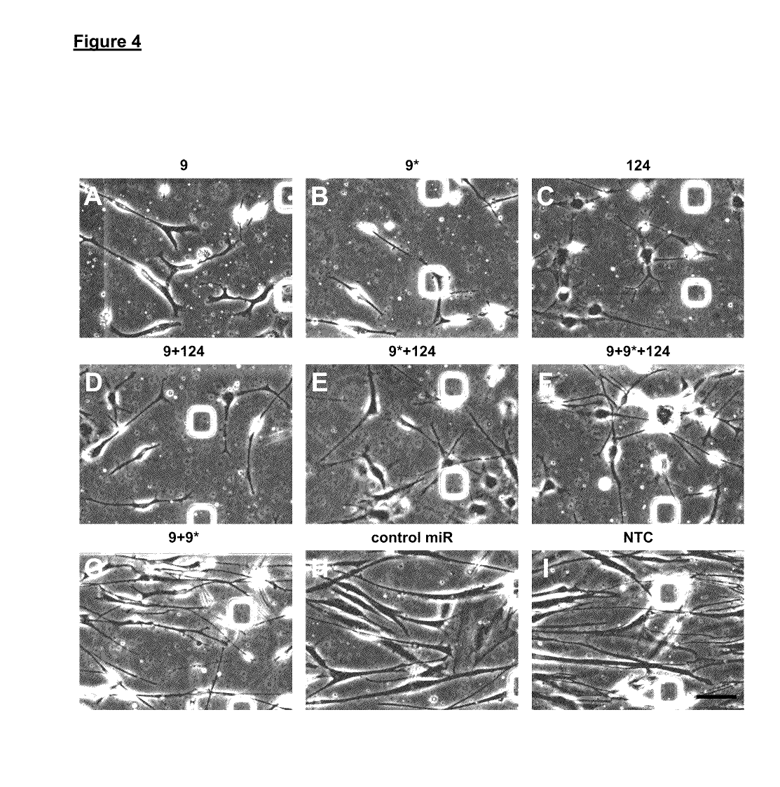

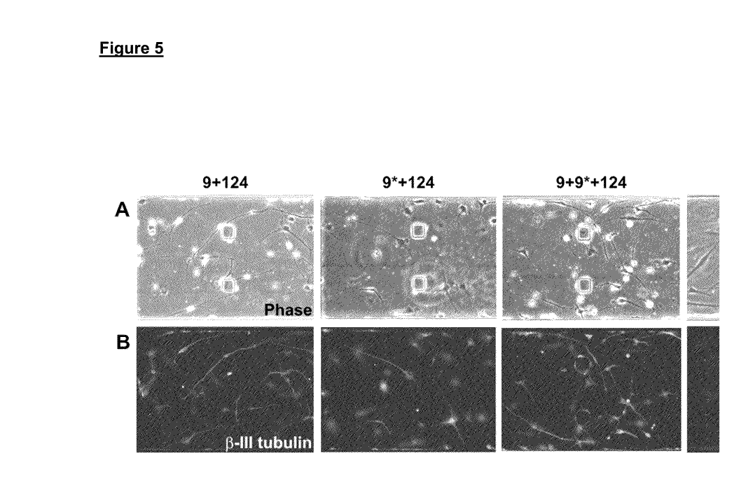

FIG. 4 is a series of phase-contrast micrographs showing the morphology of cells at the end of the protocol treated with one or two of the microRNAs alone, compared with the combination of all three. The morphology of the cells transdifferentiated using all three microRNAs shows clear evidence of neural processes, compared with the spindle-shaped fibroblast morphology shown in the controls.

MicroRNA-124 alone induced neural morphology change and inhibited cell division as early as day 2 (C); microRNA-9 and 9* also slowed down cell proliferation and induced subtle morphology changes (A, B, G). The combination of microRNA 9/124 (D), or microRNA 9*/124 (E) was enough to convert cells to neuronal-like morphology, similarly to miR 9/9*/124 (F). In contrast, FAM-labeled negative control microRNA had no effect (H, I).

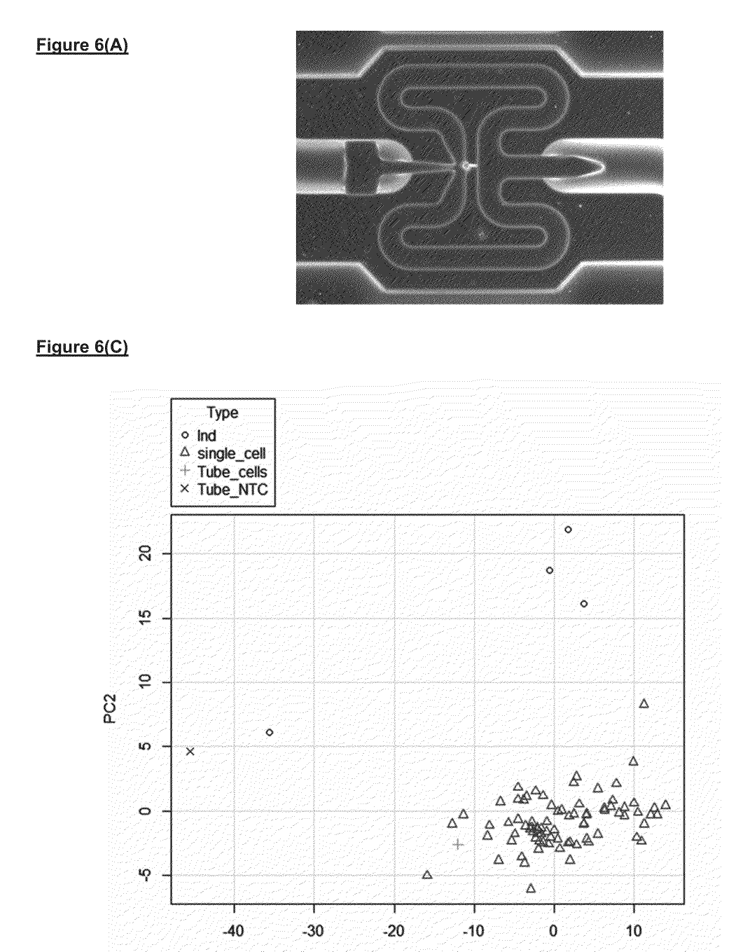

FIG. 5 compares phase-contrast images of cells treated with different microRNA combinations with the same field with immunohistochemistry staining for beta-III tubulin. To confirm the identities of the neuronal-like cells, we fixed and stained cells on chip with antibody against neuronal-specific beta-III tubulin at 8 to 24 days after the initial transfection. Results demonstrated that the microRNA combination treated-cells expressed a high level of beta-III tubulin protein from day 8. Scale bar: 100 mm.

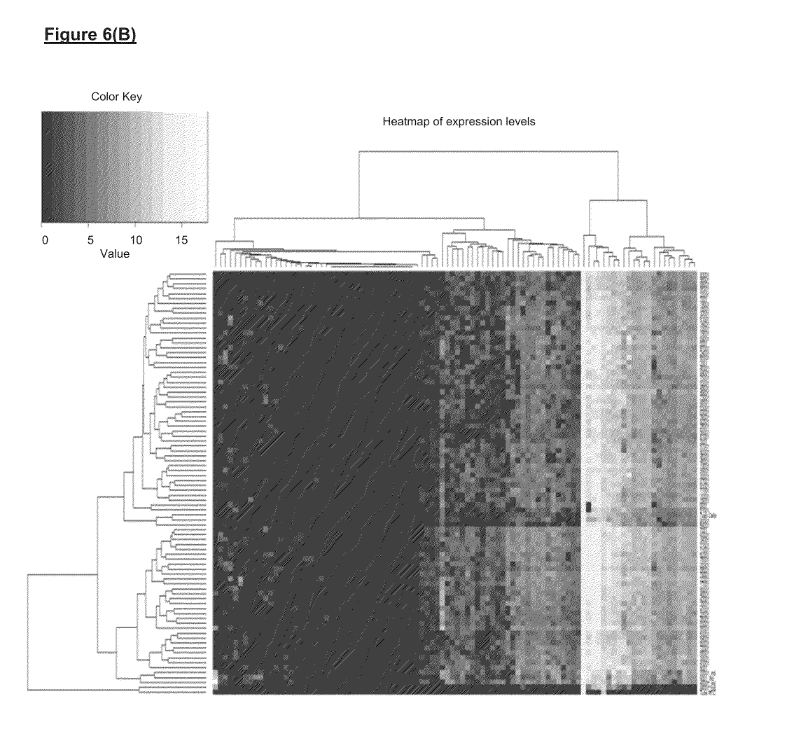

FIG. 6 shows gene expression profiles of the microRNA-treated cells. FIG. 6(A) shows a single-cell capture site (A) on a Fluidigm C1 Single-cell AutoPrep Array IFC chip for further single-cell gene expression profiling using the Fluidigm BioMark™ HD instrument.

FIGS. 6(B) and 6(C) show the analysis results of a gene expression experiment, looking at broad gene expression pattern of each cell sample. The mRNA transcript expression level of specified genes was determined using real-time RT-PCR, and obtained a CT value for each genes in each samples (single cell in this experiment). Control cells and induced cells were loaded into two different C1 chips in this experiment and data were pooled together for analysis. The CT (cycle threshold) is defined as the number of cycles required for the fluorescent signal to cross the threshold (i.e., exceeds background level). CT levels are inversely proportional to the amount of target nucleic acid in the sample (i.e., the lower the CT level the greater the amount of target nucleic acid in the sample). These methods were used to analyze the CT values to find the gene expression patterns in different samples or cells. FIG. 6(B) is the cluster analysis result; FIG. 6(C) is the principal component analysis result (PC1 and PC2 being principal components 1 and 2)

The data show high levels of neuronal-specific mRNAs such as DCX, MAP-2, NCAM1, SCN1A, TUBB3 expressed in the treated cells. The microRNA-124 target PTBP-2 was highly expressed, whereas PTBP-1 was lowered in induced neurons. Principle components analysis clearly distinguished the three microRNA-treated (“ind” as “induced”) from the control cells.

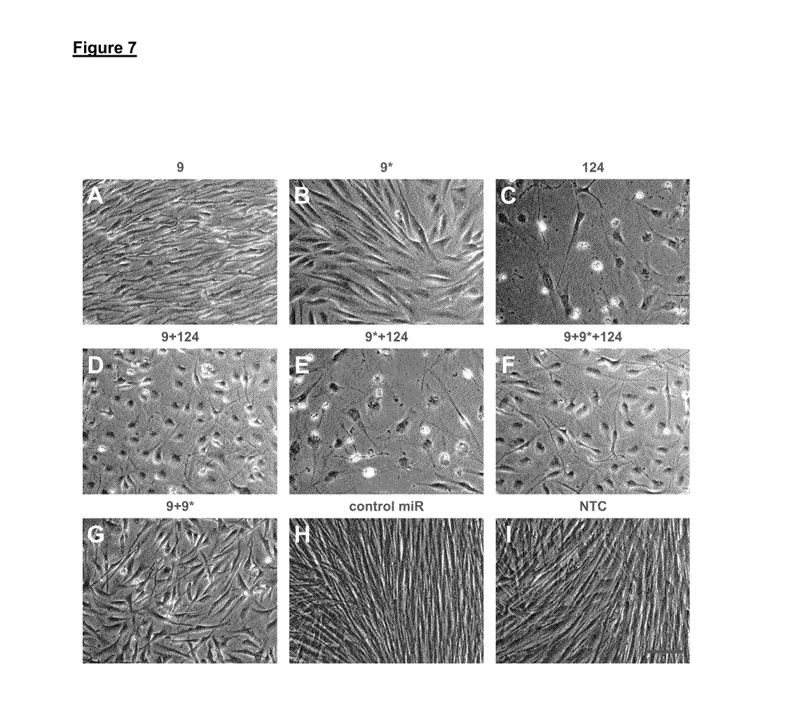

FIG. 7 is a series of phase contrast images showing combinatorial effects of microRNAs in large 12-well format. The results of the multi-factorial on-chip experiments were further confirmed in larger-format experiments in 12-well dishes. The microRNA-9 and 9* induced subtle morphology changes and slowed down cell proliferation (A, B, G). MicroRNA-124 alone greatly inhibited cell division and induced neural morphology change (C). The combination of microRNA 9/124 (D), or microRNA 9*/124 (E), microRNA 9/9*/124 (F) converted cells into neuronal-like morphology; whereas FAM-labeled negative control microRNA had no effect (H, I). Images above were acquired at day 8 post-transfection. Scale bar: 100 mm.

### Features of the Transdifferentiation Protocol

In general terms, this invention provides a method for transdifferentiating a cell from a first cell type to a second cell type. The cell is cultured or maintained in medium containing an oligonucleotide composition comprising one or more vector-free gene regulator oligonucleotides (e.g., microRNAs) so that the cell is transdifferentiated from the first cell type to the second cell type. The conditions are chosen to allow the cell to perform its standard metabolic function, and may be optimized so that the differentiation factors may promote the desired change in phenotype.

Particular gene regulator oligonucleotides are provided in this disclosure by way of illustration. The skilled reader may test possible microRNA, other regulator oligonucleotides, other differentiation factors, and combinations thereof and optimize the conditions for their use using the screening devices and protocols outlined earlier in this disclosure.

The cells may be contacted with the oligonucleotide (e.g., microRNA) containing composition (e.g., culture media containing miRNA(s)) one or more than once to effect transdifferentiation, such as 2, 3, 4, 5, 6, 7, 8, 9 or 10 times, or at least 2, 3, 4, 5, 6, 7, 8, 9 or 10 times. Separate periods of contacting (which can be referred to as “transduction periods”) are typically, but optionally, separated by washing or culturing in the absence of oligonucleotide (“intervening periods”). Transduction periods can range in duration from 5 minutes to 10 hours, more often 0.5 hours to 5 hours, often 1 hour to 4 hours. Different transduction periods may have the same or different lengths. Intervening periods can range in duration from 1 minute to 10 hours, more often 0.5 hours to 5 hours, often 1 hour to 4 hours. Different intervening periods may have the same or different lengths. The entire culture period to detection of transdifferentiated cells may be at least 2, 4, 7, 10, 14, 21, or 28 days, often between 2 and 16 days. The progress of the cells through the transdifferentiation process can be monitored on an ongoing basis by viewing cell morphology and/or by sampling the cells in each reaction chamber and determining gene expression by the sampled cells.

The efficiency of the transdifferentiation process may be determined by assessing the proportion of cells present in the original cell population that had the morphology or marker profile of the original cell type that were transdifferentiated into cells having the morphology or marker profile of the second cell type upon completion of the protocol, or at an intermediate stage. Depending on the factors and conditions used and the change desired, the proportion of cells that transdifferentiate into the target cell type may be at least 0.1%, at least 1%, at least 2%, at least 5%, at least 10%, at least 20%, substantially a majority, or substantially all of the original cells or their progeny.

The efficiency improvement provided by the invention is especially important when the transdifferentiation is done on a small scale in a microfluidic device, and/or as part of a screening or optimization protocol. Low efficiency reprogramming or transdifferentiation may be of academic interest in some contexts, but the results of transdifferentiation will be easily missed in a small scale reaction, because the total number of cells representing a rare event in a small population will be difficult to detect: particularly if the results are assayed by sampling individual cells from the small cell population on a periodic basis. In addition, rare events have less commercial applicability in production-scale medical applications. The user may optionally subject the products of a transdifferentiation reaction to a separation protocol to harvest the transdifferentiated cells of the desired phenotype and eliminate cells having the original cell type or another type. However, scale-up and production costs are better if transdifferentiation along the desired pathway occurs with the improved efficiency provided by this invention.

Another benefit of using a vector-free oligonucleotide composition is that the user has precise control over the timing of the effects of the oligonucleotide. Thus, once the oligonucleotide has had its desired effect, it may effectively be withdrawn by washing the cells with oligonucleotide free medium. This prevents additional oligonucleotides from entering the cells, and the oligonucleotides already inside will stop having an effect in accordance with their biological half-life. Thus, the user may titrate the timing of an oligonucleotide by repeatedly administering it and washing it away at regular intervals, determining the effect after each administration.

Some transdifferentiation protocols may comprise a plurality of transformation or programming steps. In this example, a cell of a first type may be transdifferentiated into one or more intermediate cell types which are more easily transdifferentiated into the cell type that is ultimately desired.

Thus, one of the embodiments of the invention is a differentiation protocol that comprises administering a first oligonucleotide composition to the cell, said composition comprising a first vector-free gene regulator oligonucleotide; culturing the cell in the presence of the first oligonucleotide composition; withdrawing the first oligonucleotide composition from the cell; administering a second oligonucleotide composition to the cell, said composition comprising a second vector-free gene regulator oligonucleotide that was not present in the first composition; and then culturing the cell in the presence of the second oligonucleotide composition. The process may be repeated through two, three, or more than three iterations to take the cell population through two, three, or more than three intermediates to attain the final cell type desired.

Culturing of cells with a transdifferentiation oligonucleotide or other agent according to this invention may occur in a tissue culture plate or in wells of a microtiter plate. The cells may be cultured in chambers of a microfluidic device. The volume of individual chambers in which cells are cultured and exposed to transdifferentiation factors may be less than about 200, 100, 60, 30, or 10 nL; typically between about 600 and 6 nL, typically about 60 nL. In a multilayer device wherein the chambers or reaction sites are formed between layers of about 60 microns, the footprint will be less than about 20, 10, 5, 2, 1, 0.5, 0.2, or 0.1 square millimeter, typically between about 10 and 0.1 square millimeter, or about 1 square millimeter (for example, 2 mm by 0.5 mm) Depending on chamber size, cell size, and culture characteristics, the plurality of cells from a first cell type cultured in each chamber (prior to the exposure to transdifferentiation factors or oligonucleotides) may be less than about 50,000 cells, sometimes less than about 10,000 5,000, 1,000, 500, 100, 50, or 10 cells.

### Use in Regenerative Medicine

Cells that have been transdifferentiated or otherwise processed according to this invention can be used for any suitable research or commercial purpose: for example, for disease modeling, drug discovery, gene therapy, regenerative medicine, and for further development of transdifferentiation technology.

This invention addresses a need in regenerative medicine by providing a potentially reliable and consistent source of tissue for transplantation. Regenerative medicine is a therapeutic approach for a person has a physiological deficiency, and is in need of cells or tissue to remedy or supplement the deficiency. Using the technique of transdifferentiation, cells needed for therapy that are difficult to obtain or culture can be manufactured from another cell type that is easier to obtain, and which can be made to proliferate in culture.

Once the cells are transdifferentiated are cultured according to this invention, they are harvested, and optionally cultured or treated to increase yield or otherwise prepare them for use in therapy. The cells are rendered suitable for use in therapy by ensuring they are substantially free of other biological material, other cell types, viruses, and compounds that may have been present during culturing. They are then combined with a pharmaceutically compatible excipient or diluent before administration, or grown on a scaffold to assemble a tissue or stimulate physiological function of the target tissue.

Dispersed cell populations or cells assembled into tissues may be administered at or near a particular location in the body in need of the therapy, or otherwise suitable location. Dispersed cell populations may also be administered intravenously.

Suitable cells and conditions include but are not limited to neural cells in treatment of a nervous disorder, cardiac cells in the treatment of heart disease, osteoblasts in the treatment of bone fractures, pancreatic b cells in the treatment of diabetes, hepatocytes in the treatment of liver disease; nephrons in the treatment of kidney disease, hematopoietic cells line the treatment of anemia or immunodeficiency, odontoblasts in the treatment of dental conditions, retinal cells in the treatment of visual impairment, dendritic cells in the preparation of immunogenic compositions, glial cells in the treatment of spinal cord injury, endothelial and smooth muscle cells in the treatment of vascular injury, and chondrocytes in the treatment of soft tissue injury. The selection, dosage, administration schedule, and monitoring of patients is the responsibility of the managing clinician.

### Glossary

A “vector free” oligonucleotide has the normal meaning in the art. A vector free oligonucleotide is an oligonucleotide that is not in the form of a virus (such as a lentivirus, retrovirus, adenovirus, or adeno-associated virus) and does not contain any viral components for gene replication, reverse transcription, or integration into host DNA.

“Differentiation” is the process by which a cell changes phenotype, function, and/or cell markers from a cell that is a precursor and/or has replicative capacity to a more mature and/or non-replicative cell.

“De-differentiation” is a process by which a cell changes phenotype, function, and/or cell markers from a mature or terminally differentiated cell to a precursor cell, multipotent cell, pluripotent cell, or stem cell. Definition, markers, and culture systems for pluripotent cells are described in U.S. Pat. Nos. 7,153,650 and 6,800,480, which are hereby incorporated herein by reference in their entirety for all purposes.

“Transdifferentiation” is a process by which a cell changes phenotype, function, and/or cell markers from one mature cell type or cell lineage to another: for example, from a fibroblast to a neuron, hematopoietic cell, adipocyte, hepatocyte, epithelial cell, endothelial cell, chondrocyte, dendritic cell, muscle cell, cardiac cell, osteoblast, or between any of these cell types in any combination.

A “differentiation factor” is a protein or other gene product that promotes or inhibits differentiation, de-differentiation, or transdifferentiation. A “differentiation reagent” is a differentiation factor or other ingredient of a culture medium that promotes or inhibits differentiation, de-differentiation, or transdifferentiation.

A “gene regulator oligonucleotide” is a nucleic acid or nucleic acid analog that specifically increases or decreases expression of one or more genes at the level of transcription or translation once transfected into a cell. This includes but is not limited to mRNA encoding one or more differentiation factors, and also includes RNAi, siRNA, oligonucleotide decoys and microRNA that inhibit transcription or translation of one or more differentiation factors.

As used in this disclosure, the sentence structure “at least X, Y, or Z” means “at least X, at least Y, or at least Z.”

A “patient” or “subject” in the context of medical therapy is a human or other mammal in need of or worthy of treatment with transdifferentiated cells or tissues according to this invention.

In reference to therapeutic compositions, a “tissue” is an assembly of cells that are transplanted or administered to a patient without completely disassembling the assembly. A “target tissue” in regenerative medicine is a tissue in a human or mammalian subject that is in need of treatment to regain or supplement an important physiological function.

The invention has been described and illustrated in this disclosure with reference to particular embodiments for the benefit and convenience of the reader. The devices, compositions, methods, and technology of the invention may be substituted and adapted for use in different contexts for different objectives using different materials, elements, and steps without undue experimentation, thus achieving any or all of the benefits of the invention without departing from the scope of what is claimed.

In the United States of America and elsewhere as permitted by law, each publication and patent document cited in this disclosure is incorporated into the disclosure by reference in its entirety for all purposes, to the same extent and effect as if each such publication or document was explicitly and individually indicated to be incorporated by reference.

The invention claimed is:

1. A method for transdifferentiating a cell from a first cell type to a second cell type, comprising culturing the cell in medium containing an oligonucleotide composition comprising one or more vector-free gene regulator oligonucleotides so that the cell is transdifferentiated from the first cell type to the second cell type.

2. The method of claim 1, wherein the oligonucleotide composition comprises one or more vector-free microRNA(s), and a messenger RNA encoding a differentiation factor.

3. The method of claim 1, wherein the oligonucleotide composition comprises the microRNAs miR-9, miR-9*, and miR-124.

4. The method of claim 1, performed in a microfluidic device and comprising the following steps:
a) administering a first oligonucleotide composition to the cell, said composition comprising a first vector-free gene regulator oligonucleotide;
b) culturing the cell in the presence of the first oligonucleotide composition;
c) withdrawing the first oligonucleotide composition from the cell;
d) administering a second oligonucleotide composition to the cell, said composition comprising a second vector-free gene regulator oligonucleotide that was not present in the first composition;
e) culturing the cell in the presence of the second oligonucleotide composition;
f) assessing morphology and/or gene expression of the cell after transdifferentiation;
g) harvesting the cell from the microfluidic device after transdifferentiation; and
h) culturing the cell outside the device after harvesting.

5. The method of claim 4, comprising assessing expression of a plurality of markers selected from NCAM1, DCX, MAP-2, TUBB3, SCN1A, PTBP-2, and PTBP-1.

6. The method of claim 4, which is a method for transdifferentiating a population of fibroblasts such that at least 80% of the fibroblasts are transdifferentiated into neural cells.

7. The method of claim 4, comprising:
i) administering an oligonucleotide composition to the cell, said composition comprising a vector-free gene regulator oligonucleotide;
ii) culturing the cell in the presence of the oligonucleotide composition administered in step i); and then
iii) withdrawing the oligonucleotide composition administered in step i);
wherein steps i), ii) and iii) are performed in sequence for at least three iterations, each iteration optionally interspersed by a step in which the cell is cultured with media that contains no oligonucleotide.

8. The method of claim 4, wherein the cells are caused to transdifferentiate without exposure to an agent that stimulates cell division or proliferation.

9. The method of claim 4, wherein one or more microRNAs in the first oligonucleotide and/or the second oligonucleotide composition are microRNAs that stop cell division and inhibit cell proliferation.

10. A method for evaluating conditions for transdifferentiating a cell from a first cell type to a second cell type in a microfluidic device, the method comprising:
a) loading a plurality of cells into separate reaction sites of a microfluidic device;
b) simultaneously culturing the cells in the separate reaction sites under culture conditions that vary among the reaction sites in one or more parameters over a predetermined range; and then
c) determining after step b) that cells in at least one of the reaction sites have transdifferentiated from the first type to the second type by assessing both cell morphology and gene expression.

11. The method of claim 10, wherein step b) comprises simultaneously culturing the cells in the separate reaction sites under culture conditions that vary among the reaction sites with respect to two or more parameters over a predetermined range; wherein the first parameter is concentration of a first gene regulator microRNA, and the second parameter is concentration of a second gene regulator microRNA.

12. The method of claim 11, wherein said microRNA(s) are selected from miR-9, miR-9*, miR-124, mi-R1 miR-21, miR-22, mi-R23, miR-122, miR-122a, miR-148, microRNAs in the let-7b family, and any combination thereof.

13. The method of claim 10, wherein the reaction sites are arranged in the microfluidic device in a grid pattern such that one of the parameters is varied in each row of the grid, and a second of the parameters is varied in each column of the grid.

14. The method of claim 10, wherein most of said reaction sites contains a single cell.

15. The method of claim 10, comprising assessing expression a plurality of markers, some of which are specifically expressed on fibroblasts and some of which are specifically expressed on neural cells.

16. The method of claim 10, wherein the microfluidic device comprises a multiplexer that is configured such that different reagents can be administered to cells in different reaction sites at different times.

17. A microfluidic device configured to identify or verify reagents that transform cells of a first type into cells of a second type in a multi-step transdifferentiation protocol, the device comprising a plurality of units, each unit comprising:
(a) a chamber configured for cell culture;
(b) a means for introducing one or more cells into the culture chamber;
(c) a means for introducing a first composition to cells in the culture chamber;
(d) a means for withdrawing the first composition from the culture chamber;
(e) a means for introducing a second composition to cells into the culture chamber;
(f) a means for withdrawing the second composition from the culture chamber; and
(g) a means for analyzing and/or recovering cells in the culture chamber after culturing;
wherein the units are interconnected so that different first compositions and different second compositions can be combinatorially introduced into the culture chambers.

18. The microfluidic device of claim 17, wherein the units are arranged in a grid pattern such that a culture additive or parameter can be varied in each row of the grid, and a second culture additive or parameter can be varied in each column of the grid.

19. The microfluidic device of claim 17, wherein a plurality of the culture chambers contain cells, and means (c) and means (e) are each fluidly connected to a separate reservoir containing a first and a second composition, respectively,
wherein the first and the second compositions each comprise one or more microRNA differentiation factors.

20. The microfluidic device of claim 17, configured for optically assessing morphology of cells in each of the culture chambers.


SEQUENCE LISTING (3 sequences)

SEQ ID NO: 1 - RNA, 23 nt, Homo sapiens
ucuuugguuaucuagcuguauga

SEQ ID NO: 2 - RNA, 22 nt, Homo sapiens
auaaagcuagauaaccgaaagu

SEQ ID NO: 3 - RNA, 20 nt, Homo sapiens
uaaggcacgcggugaaugcc